(19) United States
(12) Patent Application Publication
(10) Pub. No.: US 2026/0146935 A1
(43) Pub. Date: May 28, 2026

(54) METHOD AND DEVICE FOR PARTICLE AND GAS DETECTION

(71) Applicant: BEAMONICS AB, Lund (SE)

(72) Inventors: Jesper BORGGREN, Lund (SE); Can XU, Lund (SE); Magnus BIRCH, Lund (SE); Kristian BERGFORS, Hjärup (SE)

(73) Assignee: BEAMONICS AB, Lund (SE)

(21) Appl. No.: 19/122,533

(22) PCT Filed: Oct. 5, 2023
(86) PCT No.: PCT/SE2023/050995

§ 371 Date: Apr. 18, 2025

(30) Foreign Application Priority Data
Oct. 21, 2022 (SE) 2230340-8

(51) Int. Cl. G01N 15/075; G01N 33/00

(52) U.S. Cl. CPC G01N 15/075; G01N 33/0027

(57) ABSTRACT

A device for detecting a property of a gas comprising a light source configured to emit a light along at least a transmission axis. The device comprises a light detection arrangement comprising: a light sensor configured to output a sensor signal and a lens arrangement having a lens plane, and being configured to direct the light from the light source and scattered by the gas to the light sensor. The device also comprises an actuator assembly configured to move the light sensor in a direction parallel to at least a first axis. The first axis, the transmission axis, and the lens plane intersect such that a Scheimpflug condition is achieved.

### FIELD

The present disclosure relates to laser projection systems and more particularly to systems for detecting gases and properties of gases.

### BACKGROUND

A LIDAR or laser radar is an optical device for detection and ranging with applications in a very broad range of environments, from industrial combustion furnaces to ecosystem monitoring. In contrast to the now wide-spread topographical LIDAR systems which detect and range hard targets, atmospheric LIDARs have sufficient sensitivity to retrieve a continuous molecular echo from entirely clean air.

A highly specific atmospheric LIDAR method is the Differential Absorption LIDAR (DIAL). In this method, a pulsed tuneable laser targets specific molecular absorption lines and concentration profiles of a gas can be acquired. In practice, high peak powers (MW), short pulses (ns), narrow bands (<pm) and tunability contradict each other. Such DIAL systems typically require a small team of PhDs in laser physics to run. Some progress has been reported on lighter and smaller DIAL systems using micro-LIDAR, but still with time resolution in the order of 10 minutes. Consequently, DIAL systems are immensely expensive and there are only a handful operational on a global basis. The low resolution, the cost and the bulkiness of DIAL systems prevent many practical applications such as industrial process optimization and mapping of greenhouse gas sources and fluxes.

A known alternative LIDAR method is disclosed in U.S. Pat. No. 11,169,272. This discloses an optical arrangement for using the Scheimpflug condition for analysing gas absorption lines at different distances within the same field of view. A problem with this arrangement is that the system requires a complex optical sensor arrangement suitable for focusing and sampling received light corresponding to different distances. Such a system in addition further needs fulfilments of a number of conditions, e.g., Scheimpflug condition and Hinge rule during operation, with very limited flexibility during operation and parameters are not easily changed during runtime due to the requirement of the optical conditions to fulfil. Furthermore, this type of array or 2D imaging sensor, coupled with speed requirements of kHz, in particular for wavelengths longer than silicon wavelength can be immensely expensive. Furthermore, such an arrangement typically poses non-trivial requirements on the sensor assembly in terms of speed as well as the processing unit due to its inherent coupling to imaging sensors and processing. This means that installation, operation, and maintenance can be complex and time consuming.

### SUMMARY

Examples of the present disclosure aim to address the aforementioned problems.

According to an aspect of the present disclosure there is a device for detecting a property of a gas comprising: a light source configured to emit a light along at least a transmission axis, a light detection arrangement comprising: a light sensor configured to output a sensor signal, a lens arrangement having a lens plane, and being configured to direct the light from the light source and scattered by the gas to the light sensor, an actuator assembly configured to move the light sensor in a direction parallel to at least a first axis, wherein the first axis, the transmission axis, and the lens plane intersect such that a Scheimpflug condition is achieved.

Optionally, the actuator assembly is further configured to move the light sensor in a direction parallel to a second axis, corresponding to an optical axis of lens arrangement.

Optionally, the actuator assembly is further configured to move the light sensor in a direction parallel to a third axis orthogonal to the second axis and in a plane defined by the first axis and second axis.

Optionally, the actuator assembly is configured to move the light sensor in a direction parallel to a fourth axis along a normal of the plane defined by the first axis and second axis.

Optionally, the light sensor is mounted on a sensor assembly.

Optionally, the light sensor is moveable with respect to the sensor assembly and the actuator assembly comprises a first actuator configured to move the light sensor.

Optionally, the sensor assembly is moveable with respect to a housing of the device and the actuator assembly comprises a second actuator configured to move the sensor assembly.

Optionally, the sensor assembly is moveable with respect to a housing of the device along a rail.

Optionally, the light sensor comprising at least one of a single pixel, a quadrant of pixels, an array of pixels, a pixel matrix, a position sensitive device (PSD) pixel.

Optionally, the light sensor comprising at least one column of pixels aligned parallel to the first axis.

Optionally, the light sensor comprising at least one row of pixels aligned parallel to the fourth axis.

Optionally, the light sensor comprises at least one of a photo diode, an avalanche photodiode, a photo multiplying tube (pmt), and a cmos sensor.

Optionally, the light sensor comprises at least one of a trans-impendence amplifier, free silicon amplifier, current amplifier, and dynode amplifier.

Optionally, the light sensor being configured to detect a signal generated by at least one of wavelength modulation spectroscopy, direct absorption spectroscopy, and/or frequency modulation spectroscopy.

Optionally, light source is a tuneable diode laser.

Optionally, the light source is controlled in a TDLAS fashion.

Optionally, the light source is controlled in a DIAL fashion.

Optionally, light source comprises an array of individual light sources arranged parallel to a fifth axis orthogonal to transmission axis or parallel to the fourth axis.

Optionally, the light sensor comprises a single sensor pixel.

Optionally, the device comprises a sensor window positioned between the light sensor and the lens arrangement.

Optionally, the sensor window comprises a slit having adjustable width in a direction along the first axis.

In another aspect of the disclosure, there is provided a method for detecting a property of a gas comprising: emitting a light along at least a transmission axis, directing the light scattered by the gas to a light sensor using a lens arrangement having a lens plane, imaging a volume of gas at a particular distance from the device by moving the light sensor to a corresponding position along a first axis, wherein the first axis, the transmission axis, and the lens plane intersect such that a Scheimpflug condition is achieved.

### BRIEF DESCRIPTION OF THE DRAWINGS

These and other aspects, features, and advantages of which examples of the disclosure are capable of will be apparent and elucidated from the following description of examples of the present disclosure, reference being made to the accompanying drawings, in which;

FIG. 1 shows a schematic plan diagram of a device according to an example of the disclosure;

FIG. 2 shows a diagram of an application according to an example of the disclosure;

FIG. 3 shows a schematic plan diagram of a device according to an example of the disclosure;

FIG. 4 shows a diagram of a device according to an example of the disclosure;

FIGS. 5a and 5b show respectively a schematic plan diagram of a device according to an example of the disclosure;

FIG. 6 shows a schematic perspective diagram of a device according to an example of the disclosure;

FIGS. 7a, and 7b show a close-up of part of a device according to an example of the disclosure;

FIG. 8 shows another close-up of part of a device according to an example of the disclosure;

FIGS. 9a and 9b show another close-up of part of a device according to an example of the disclosure;

FIG. 10 shows a flow diagram of a method according to an example of the disclosure.

### DETAILED DESCRIPTION

In the following, examples of the present disclosure will be presented for a specific example of a gas sensing and particle sensing device 100. Throughout the description, the same reference numerals are used to identify corresponding elements.

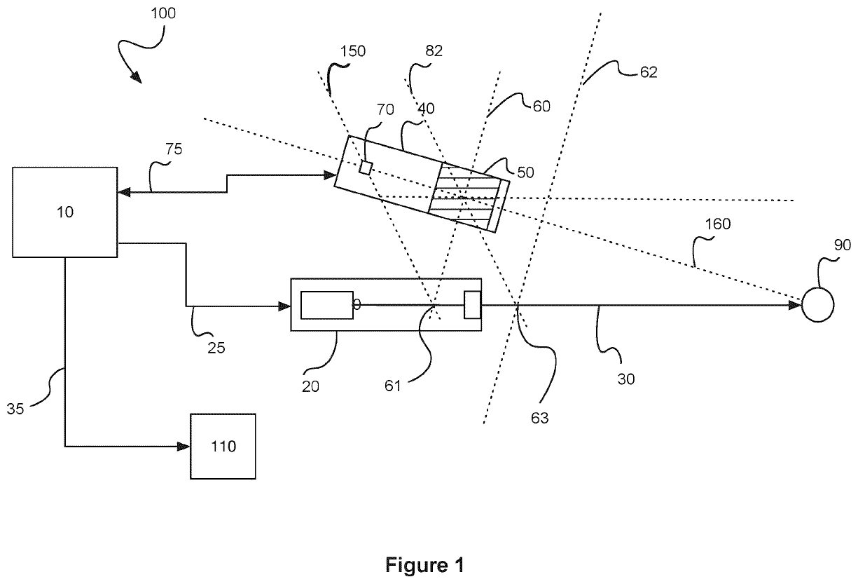

FIG. 1 shows an embodiment of the device 100. In some examples, the device 100 is a gas detecting device 100, in some examples, the device 100 is a particle 90 detecting device 100. The device 100 is configured to direct light from a light source 20 and receive scattered light from one or more particles 90. The one or more particles 90 are a gas or hard matter located at a distance from the device 100. This can be both gas particles 90 and/or hard particles 90. The one or more particles 90 may be a gas such as ozone, nitric oxide (e.g., NOx), a sulphurous oxide (e.g., SOx), water, oxygen, nitrogen, hydrogen (H2), CO2, CO, CH4, Acetylene C2H2, Formaldehyde H2CO, Hydrogen Sulfide H2S, Hydrogen Chloride HCl, Ammonium NH3, Ethane, C2H6, Hydrogen fluoride HF or any other gas, naturally present or released, in the atmosphere. In some other examples, the one or more particles 90 may additionally or alternatively be a particulate pollutant e.g., dust, soot, smoke.

In some examples, the one or more particles 90 can be:
Large coarse particles 90 having a diameter greater than 10 μm;
Coarse particles 90 (also known as PM_{10-2.5}) particles 90 with diameters generally larger than 2.5 μm and smaller than, or equal to, 10 μm in diameter;
Fine particles 90 (also known as PM_{2.5}): particles 90 generally 2.5 μm in diameter or smaller;
Ultrafine and nanoparticles which have diameters less than 2.5 μm.

Typically, identification of coarse particles 90, fine particles 90, ultrafine particles 90, and nanoparticles is desirable because these particles 90 can enter the lung and damage respiratory systems. Furthermore, even disregarding the influence of particles 90 on human health, identification and monitoring of the particles 90 is highly relevant for the industry, for example, in processing industry and clean-room environments, where particles 90 directly impact the quality of the produced goods. Particulates 90 can comprise one or more particles 90 of sulphate, nitrate, ammonium, elemental carbon, organic carbon, silicon, and sodium ions. In other examples, the particulates 90 can be any particles 90 in the atmosphere having the above-mentioned size. The measurement of particulate 90 size is well-known and will not be discussed any further.

The device 100 comprises a data processing device 10. The data processing device 10 is configured to issue control signals to one or more components of the device 100 during operation. The data processing device 10 is also configured to generate an indication whether one or more particles 90 are detected in the gas. In this way the data processing device 10 is configured to drive light source 20 and to process the sensor signal 75 to determine a property of the gas.

In some examples, the data processing device 10 may be implemented by special-purpose software (or firmware) to run on one or more general-purpose or special-purpose computing devices 10. In this context, it is to be understood that each “element” or “means” of such a computing device 10 refers to a conceptual equivalent of a method step; there is not always a one-to-one correspondence between elements/means and particular pieces of hardware or software routines. One piece of hardware sometimes comprises different means/elements. For example, a processing unit serves as one element/means when executing one instruction but serves as another element/means when executing another instruction. In addition, one element/means may be implemented by one instruction in some cases, but by a plurality of instructions in some other cases. Such a software-controlled computing device 10 may include one or more processing units, e.g., a CPU (“Central Processing Unit”), a DSP (“Digital Signal Processor”), an ASIC (“Application-Specific Integrated Circuit”), discrete analog and/or digital components, or some other programmable logical device 10, such as an FPGA (“Field Programmable Gate Array”). The data processing device 10 may further include a system memory and a system bus that couples various system components including the system memory to the processing unit. The system bus may be any of several types of bus structures including a memory bus or memory controller, a peripheral bus, and a local bus using any of a variety of bus architectures. The system memory may include computer storage media in the form of volatile and/or non-volatile memory such as read only memory (ROM), random access memory (RAM) and flash memory. The special-purpose software may be stored in the system memory, or on other removable/non-removable volatile/non-volatile computer storage media which is included in or accessible to the computing device 10, such as magnetic media, optical media, flash memory cards, digital tape, solid state RAM, solid state ROM, etc. The data processing device 10 may include one or more communication interfaces, such as a serial interface, a parallel interface, a USB interface, a wireless interface, a network adapter, etc, as well as one or more data acquisition devices 10, such as an A/D converter. The special-purpose software may be provided to the data processing device 10 on any suitable computer-readable medium, including a record medium and a read-only memory.

The discrimination of co-and de-polarized light in LIDAR may provide microstructural information about LIDAR targets. Single scattering aerosol LIDAR may be defined as a type of atmospheric LIDAR sensitive to receive echoes from clean air. In single scattering aerosol LIDAR, the depolarization ratio (DoLP), which is defined as the intensity ratio between the perpendicular component and the parallel component of the scattered light, can differentiate between spherical and edgy or irregularly shaped particles 90 such as droplets and ice crystals respectively. Furthermore, particle 90 sizing can be determined by means of discrimination of signals from either several wavelengths or based on the signal intensity with a-priori knowledge of the particulate distribution in the gas.

The data processing device 10 is configured to send a control signal 25 to a light source 20 so that the light source 20 emits light along a transmission axis 30.

The light emitted from the light source 20 travels along the transmission axis 30 until it reaches a particle 90 in the atmosphere. At least some of the emitted light from the light source 20 is scattered back towards a light detection arrangement 40 from the particle 90 along a second axis 160. The second axis 160 is a received light path axis 160 e.g., the path the scatter light takes from the particle 90 to the device 100.

The light detection arrangement 40 comprises a lens arrangement 50 having a lens plane 60. The lens arrangement 50 as shown in FIG. 1 is aligned along the second axis 160. Whilst the second axis 160 is one backscattering axis in practice there may be a plurality of different backscattering axes from different particles 90 at different distances from the light detection arrangement 40. The lens arrangement 50 is configured to direct the light scattered by the scattering particle 90 to a light sensor 70.

In one embodiment, the light sensor 70 has a pixel column aligned to an image plane and configured to output a sensor signal 75 to the data processing device 10. The image plane may be aligned along a first axis 150. The first axis 150, the lens plane 60, and the transmission axis 30 intersect such that a Scheimpflug condition is achieved at first intersection 61.

Furthermore, in some examples, a displaced image plane 82, a front focal plane 62 of the lens arrangement 50, and the transmission axis 30 of the light source 20 optionally fulfil the Hinge rule at second intersection 63. The data processing device 10 processes the sensor signal 75 from the light detection arrangement 40 to determine a pixel signal for one or more pixels of the light sensor 70. The fulfilment of the Hinge rule relationship between the light source 20 and the light detection arrangement 40 is optional.

The lens arrangement 50 may comprise at least one of: an imaging lens comprising one or more light refracting components, and a mirror lens comprising a catadioptric optical system. The lens arrangement 50 comprises an f-number F/#, aperture, ø_{rec} and focal length, f_{rec}. The lens arrangement 50 may further comprise of one or optical filters. In some examples, the optical filters comprise of interference-type optical filters, in some examples the optical filters comprise of color coated optical filters, the optical filters could be of bandpass type, short-pass, long-pass or a combination of the types.

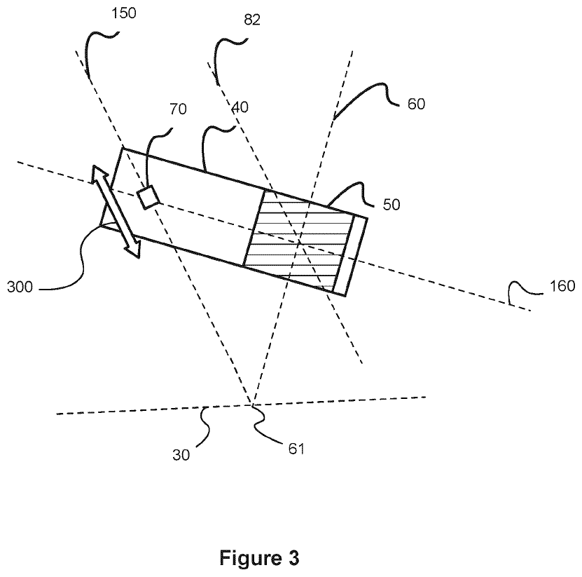

The light sensor 70 is a CMOS array detector or an array detector with low number of pixels, or a single-pixel detector, such as a photodiode. By providing a light sensor 70 with a single pixel, the light sensor 70 is less complex. The light sensor 70 is optionally mounted behind a sensor window 702. Furthermore, the pixels of the light sensor 70 are smaller which improves resolution. The light sensor 70 is moveable and movement of the light sensor 70 will be discussed in more detail below in reference to FIGS. 3, 4, 5a, 5b, and 6. The light sensor 70 is further configured to output a sensor signal 75. The moveable light sensor 70 has a sensor length ( [symbol] ), Sensor tilt (θ), and a number of pixels. The pixels have a pixel height ( [symbol] ), and pixel width (wₚᵢₓ).

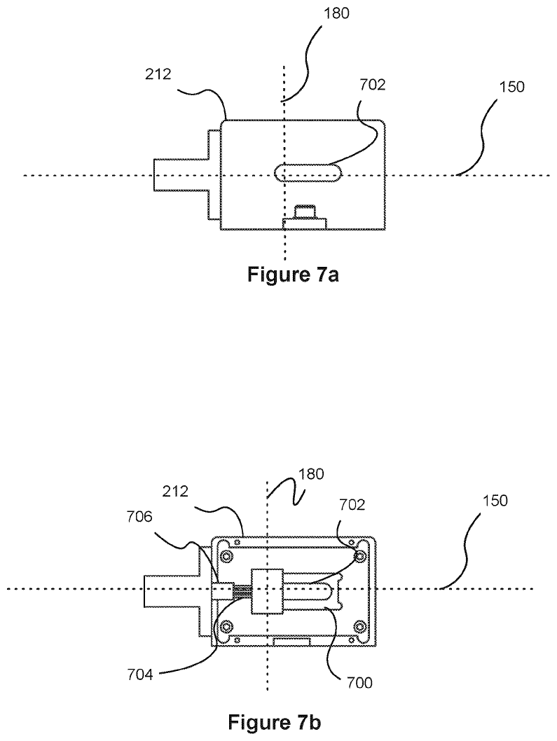

In some examples, the light sensor 70 comprises a single moveable pixel. In other examples, the height and width of the moveable pixel can be changed through one or more slits in front of the pixel and behind of the sensor windows 702. The sensor window 702 is shown in FIGS. 7a and 7b and is discussed in further detail below. The sensor window 702 can optionally comprise an adjustable slit component configured to control the scattered light received by the light sensor 70. The adjustable slit component is described in more detail below in connection with FIGS. 7a and 7b. In some other examples the light sensor 70 comprises a small array of moveable pixels. For example, the light sensor 70 comprises an array of pixels 2 by 2, 3 by 3, 4 by 4, 5 by 5 or n by n where n can be any number between 1 and 20, the height and width of the array can be changed through one or more slits in front of the pixel and behind of the sensor window 702.

In some other less preferred examples, the light sensor 70 comprises a moveable linear CMOS array detector and may comprise of at least one column of pixels aligned to an image plane aligned with the first axis 150. The moveable linear CMOS array detector light sensor 70 is further configured to output a sensor signal 75. Light sensor 70 has a sensor length ( [symbol] ), Sensor tilt (θ), and a number of pixels. The pixels have a pixel height ( [symbol] ), and pixel width (wₚᵢₓ).

After employing the Scheimpflug principle or the Scheimpflug principle and the Hinge rule, a number of design parameters remain for consideration. The device 100 may be designed with the following variables in mind: The transmitter-receiver baseline separation distance, [symbol] , the receiver focal length, f_{rec}, and the tilt of the sensor with respect to the lens plane 60, θ. The transmitter-receiver baseline separation distance is defined as the perpendicular distance between lens arrangement 50 and transmission axis 30. The receiver focal length is defined as the perpendicular distance between lens plane 60 and front focal plane 62.

The device 100 comprises an actuator assembly 110 configured to move the light sensor 70 parallel with or along a first axis 150. This means that the position of the light sensor 70 can be adjusted rather than providing a large sensor that extends across a wider area. Accordingly, the light sensor 70 can be simulated to be a larger sensor by moving the light sensor 70 with the actuator assembly 110 through a volume of space.

This makes operation of the device 100 simpler because a smaller and less complex light sensor 70 can be used. Since the light sensor 70 moves along the first axis 150, the Scheimpflug principle and the Hinge rule can be easily maintained without a complex sensor. Adjusting the lens arrangement 50 to change the focal point to the light sensor 70 makes the optical arrangement more complex and more likely to mean that the Scheimpflug principle and/or the Hinge rule cannot be maintained over a large range of configurations.

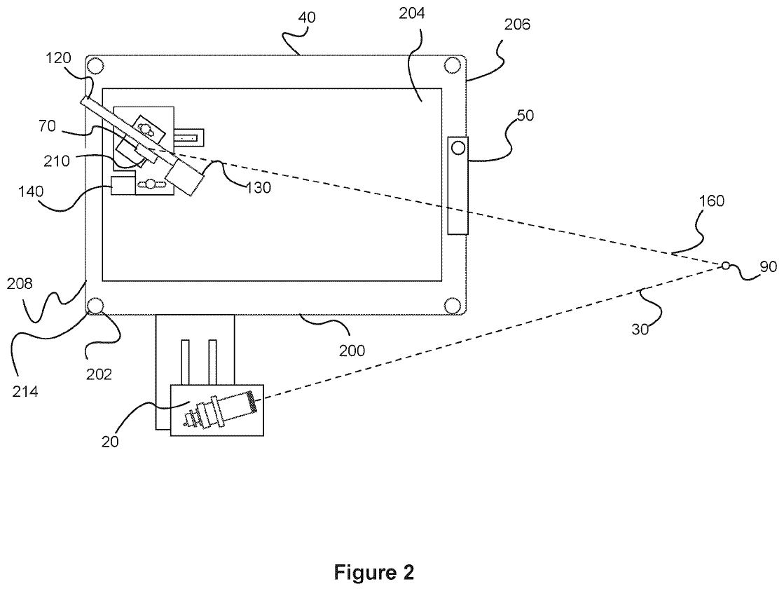

FIG. 2 shows the light detection arrangement 40 of the device 100. The light detection arrangement 40 comprises a base 200. The base 200 is a rigid structure for mounting one or more components of the device 100 thereto. The base 200 is a planar structure that extends in a plane parallel to the first axis 150 and the second axis 160. The base 200 in some examples is a metal structure or another suitable rigid structure. This means that the components mounted to the base 200 can be fixed with respect to each other to maintain the spatial and optical relationships.

The base 200 itself can optionally comprise one or more mounting holes 202 for fastening the device 100 to a work surface (not shown) or another object. Alternatively, the mounting holes 202 are for receiving a lid (not shown) for protecting the device 100 when in use. The mounting holes 202 are optionally located in the corners of the base 200 and comprise a threaded bore 706 for receiving a threaded bolt (not shown).

The base 200 optionally comprises a component mounting plate 204 configured to receive the components of device 100. The component mounting plate 204 in some examples is machined from a stiff metal plate with predetermined mounting positions for receiving the components of the device 100. In some other examples, the base 200 does not have a component mounting plate 204 and instead the components are mounted directly to the base 200.

As shown in FIG. 2, the lens arrangement 50 is mounted at a first end 206 of the base 200. As mentioned above, the lens arrangement 50 may comprise at least one of: an imaging lens comprising one or more light refracting components, and a mirror lens comprising a catadioptric optical system. The lens arrangement 50 comprises an f-number F/#, aperture, ø_{rec} and focal length, f_{rec}. The lens arrangement 50 may further comprise of one or more optical filters. In some examples, the optical filters comprise of interference-type optical filters, in some examples the optical filters comprise of color coated optical filters, the optical filters could be of bandpass type, short-pass, long-pass or a combination of the types. The lens arrangement 50 is aligned on the second axis 160.

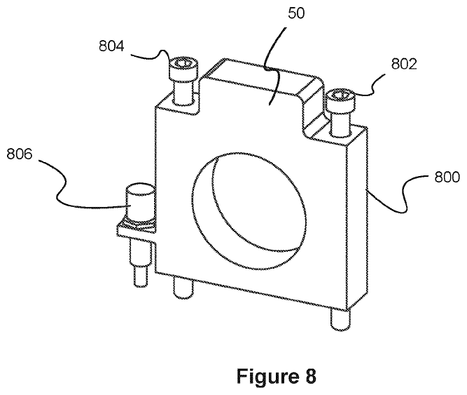

The lens arrangement 50 is also shown in FIG. 8 and will now be briefly described with respect to the Figure. Figure shows a perspective view of the lens arrangement 50.

The lens arrangement 50 comprises a lens frame 800 in which one or more lenses, one or more optical filters (not shown for the purposes of clarity) are mounted. The lens frame 800 comprises two mounting bolts 802, 804 for mounting the lens arrangement 50 to reciprocal mounting holes in the optional component mounting plate 204.

In some examples, the lens arrangement 50 comprises an adjustment mechanism 806. The adjustment mechanism 806 is configured to make fine adjustments of the position of the lens arrangement 50 with respect to the base 200 after the lens arrangement 50 is mounted to the base 200 and optionally component mounting plate 204. In some examples, the adjustment mechanism 806 is configured to move the lens arrangement 50 in a direction parallel to a fourth axis 180. The fourth axis 180 is an axis which is perpendicular to the second axis 160. In this way the adjustment mechanism 806 is configured to adjust the height of the lens arrangement 50 above the base 200. This helps align the lens arrangement 50 on the second axis 160. In some examples, means for adjusting the position of the lens arrangement 50 with respect to the base 200, such as the adjustment mechanism 806, are motorized, to enable controlled and/or automatic adjusts. In some examples the adjustment mechanism 806 is an actuator e.g. a stepper motor. However, in some other examples, the adjustment mechanism 806 is a manually adjusted adjustment screw. This allows for optimization to maximize the overlap of the imaged emitted light 30 along the second axis 160 and collected by the light sensor 70.

The position of the light sensor 70 as shown in FIG. 2 is decoupled from the position of the lens arrangement 50. This means that even if there is some misalignment of the light sensor 70, as the light sensor 70 is moved along the first axis 150, the misalignments can be corrected with the adjustment mechanism 806 of the lens arrangement 50. The misalignments can be corrected simultaneously by the adjustment mechanism 806 as the light sensor 70 is moved. This is in contrast to prior art solutions whereby in a traditional array-sensor, the positional height of a lens arrangement needs to be matched with the emitted light since the position of the lens arrangement cannot be decoupled from the position of a light sensor.

Turning back to FIG. 2, the light source 20 will now be discussed in more detail. In some examples, the light source 20 is a tuneable diode laser. The light source 20 may comprise one or more of; a narrowband single-mode source, a broad band multi-mode source, a high-power multimode diode laser, a high-power multimode fibre laser, a high-power tapered amplifier seeded by a tuneable single mode diode laser, a high-power fibre amplifier seeded by a tuneable single mode diode laser, and a high-power tuneable CO2 or solid-state crystal laser. Indeed, the light source 20 can be any suitable light source 20 for generating light to be transmitted to the gas to be analysed. In some other examples, the light source 20 can be other types of light source 20 e.g., a non-coherent light source such as an LED or incandescent light bulb.

In one embodiment, the light source 20 is a multimode 10 W, 761 nm, 2 nm FWHM (Full width at half maximum) CW (continuous wave) laser diode. The acquisition of some 400 elastic spectral bands in the range 760 nm to 762 nm is performed. This will allow the resolving of a large number of O₂ absorption lines. Whilst reference to O₂ absorption lines is made, this is exemplary and other absorption lines of other gas molecules or particulate 90 molecules may be resolved e.g., any absorption lines of the gases or light scattering properties of particulates 90 mentioned in this disclosure may be detected. The absorption lines provide information on concentration, pressure, and temperature of the air. Generally, O₂ concentration in the atmosphere is 21%, but local exhausts after metabolism or combustion can produce O₂ holes. The drop in O₂ corresponds to the rise in CO₂ and H₂O. Consequently, the drop in O₂ may provide information on, e.g., the amount of metabolism present. Alternatively, the amount of fuel consumed by an engine may be determined, providing a means for normalizing aerosol emissions, and assessing engine quality. This technique allows indirect assessment of profiling of CO2, pressure, and temperature.

To ensure a good gas sensitivity of the light source 20, the data processing device 10 is configured to one or more parameters of the light source 20. In some examples, the data processing device 10 is configured to provide fine control of the temperature, current and voltage driving properties of the light source 20. Furthermore, the data processing device 10 is configured to provide anti-interference measures of the light source 20.

In some examples, the data processing device 10 is optionally configured to provide temperature control of the light source 20 which is achieved through the use of a thermistor (not shown), e.g., Negative Temperature Coefficient (NTC) thermistor or similar, and a thermoelectric cooler (TEC) in a feedback loop. Sampling of the thermistor with high precision is well known and can be achieved through a biased scheme or unbiased scheme and sampled through analog to digital converters. The temperature can then easily be retrieved, through linearization of the voltages.

However, controlling and driving of the TEC is less trivial since large currents needs to be switched in a controlled fashion in two directions, one direction for heating, and one direction for cooling. The large currents (˜Amperes) involved and the speed required commonly result in H-bridge configurations, similar to the driving of electrical motors. This approach, however, has large drawbacks in a sensitive optical system as described in this disclosure, since the large currents involved require switching electronics which can easily induce unwanted noise caused both by the switching hardware itself as well as the large currents.

Accordingly in some examples, the data processing device 10 is optionally configured to generate and control currents through the use of buck regulators or buck converters (not shown). Such converters are usually not capable of sinking currents, making them not ideal for current-driving applications. However, in one example, one or more buck converters are used in a synchronous topology in order to both be able to sink current and to source current. By employing two such buck converters, each output voltage can be individually tuned and hence control of the direction of current flow is achieved. The benefit is the high frequency in which these devices inherently operate, which can be in the several MHz range, making them essentially quiet from the perspective of the instrument which operates at lower frequencies. This is of big advantage and importance in order to achieve a good sensitivity. Furthermore, this example also enables a smaller footprint since less components are required, in addition, the driving scheme does not inherit the necessity of dead-time-insertions as required by the well known H-bridge configurations, able to operate at 100% duty cycle providing a more energy efficient solution and noise-free solution.

In another example, the driving configurations of the light source 20 are optionally addressed. Present implementations rely often on manual or semi-automatic calibration and testing procedures by operators during productions, where the driving properties, e.g., voltages, currents, temperatures and mitigations of optical interference properties, are determined. This is entirely decoupled from the light source 20 itself, making this a cumbersome, time-consuming, costly and prone to human error process.

In this example, a storage device (not shown) is optionally integrated into the light source itself. Such a storage device could be a read-only memory, e.g., EEPROM, flash, F-RAM or other types of persistent storage devices. Alternatively, such storage device could be a read-write device with persistent storage properties. The storage device contains the model and calibration values of the light source which can be read out by the instrument to automate the testing and calibration steps of the instrument. Such information may be important for the purpose of the topic of optical gas sensing as described in this disclosure and small deviations from optimum conditions may easily reduce the quality and performance of said instrument significantly. In another example, the data processing device 10 can write data back to the storage device, and in this way potential issues identified, related to, e.g., lifetime, aging, current, voltage and temperature deviations, are logged to the light source 20 which can be used to improve processes, troubleshooting and traceability.

In another example, mitigations against optical interference may be optionally addressed. This may be very important for long-coherence-length light source 20, i.e., light sources 20 with narrow bandwidths for gas and particle sensing. In some examples, the data processing device 10 is configured to provide a form of dithering employed to average the laser speckles out, which otherwise is the dominant optical noise factor in an instrument. The dithering is achieved by movement of lenses close to the light source 20, the light source 20 itself, movements of diffusive elements, or the entire light source 20 assembly itself. This can be achieved by a motor, a piezoelectric crystal, liquid lenses or impulse devices. Driving of these devices often requires very high voltages in the range of +/−100 V. Since the voltage range is very different from the rest of the sensitive hardware, noise immunity and configurability are of big importance for design.

For example, configuration of the hardware is in principle always necessary if the load changes, or ages. In some examples a generic integrated solution is optionally provided which is able to drive loads regardless of their electrical properties, e.g., different capacitances, resistance and inductance. This is achieved by a buck-boost converter (not shown) acting as a high-voltage amplifier, such buck-boost converters have been used in, e.g., haptic devices, and provides automatic feed-back and loop for different types of loads which may also change in time simultaneously as different driving patterns are used. In some examples, this buck-boost converter driver is combined with a simple digital to analogue converter which generates an arbitrary waveform which the high-voltage buck-boost converter will output to the dithering devices, independent on the load, and self-referencing to ensure proper function and movement of the dithering device.

In some examples, the data processing device 10 is configured to send a control signal 25 to the laser diode of the light source 20 to control the laser diode with a TDLAS (tuneable diode laser absorption spectroscopy) mode of operation and or a DIAL (Differential absorption lidar) mode of operation. Both TDLAS and DIAL modes of operation are known and will not be discussed any further. In some examples, the control signal sent from the data processing device 10 to the light source 20 can be one or more of a control signal configured to control the temperature (double buck), dithering (high-voltage amplifier) and/or current driving the source (integrated data storage) as discussed above.

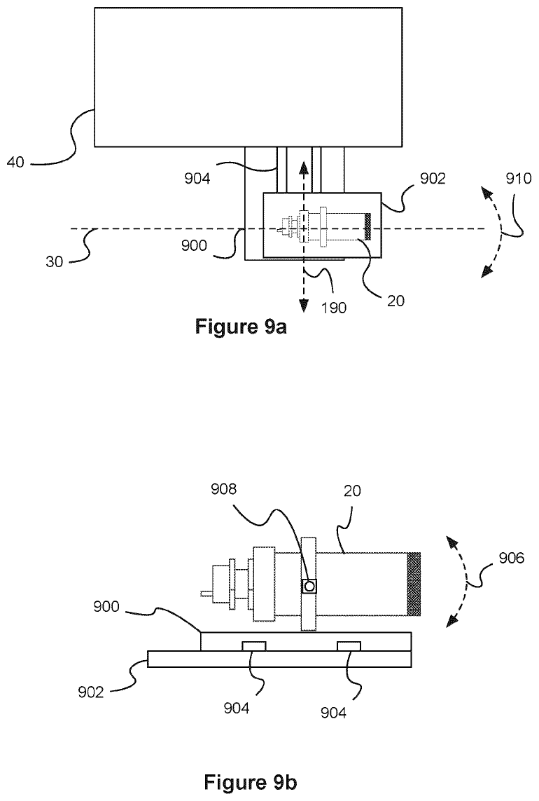

FIGS. 9a and 9b also respectively show an example of the light source 20 in a plan view and a side view.

The light source 20 in FIG. 2 is shown positioned to the side of the base 200. The position of the light source 200 is fixed with respect to the base 200 during operation. In some examples, the light source 20 is also mounted fixed to the base 200 or an accessory fixed with respect to the base 200 as shown in FIGS. 9a and 9b.

For example, the base 200 in some examples can extend underneath the light source 20 so that the light source 20 can be fixed to the optional component mounting plate 204. As shown in FIG. 9a, the base 200 of the light detection arrangement 40 is connected to a transverse rail assembly 900. The transverse rail assembly 900 is fixed with respect to the base 200. The light source 20 is mounted on a light source carriage 902 which is moveable on the transverse rail assembly 900. The light source carriage 902 is configured to be fixed with respect to the transverse rail assembly 900 with a clamp (not shown) or other suitable releasable fastening. The transverse rail assembly 900 comprises a pair of rails 904 and the light source carriage 902 slides along the rails 904. The rails 904 allow movement in a direction along a fifth axis 190 angled to the transmission axis 30. In some examples, the fifth axis 190 is orthogonal to the transmission axis 30, however in some other examples, the fifth axis 190 can be angled at any suitable angle with respect to the transmission axis 30. Movement of the light source carriage 902 along the fifth axis 190 means that the transmission axis 30 can be moved with respect to the light detection arrangement 40. The light source carriage 902 in some other examples also allows relative movement of the light source 20 and the transmission axis 30 with respect to the light detection arrangement 40 along different axes and directions. The light source carriage 902 optionally comprises a pivotal mounting 908 that permits the light source 20 to pivot in a first pivoting direction 906 with respect to the light source carriage 902. This can incline the transmission axis 30 with respect to the base 200. Additionally, or alternatively, the pivotal mounting 908 permits the light source 20 to pivot in a second pivoting direction 910 with respect to the light source carriage 902. In this way the first and second pivoting directions 906, 910 are orthogonal. The first pivotal direction 906 is orthogonal to the plane of the light source carriage 902. The second pivotal direction 910 is parallel to the plane of the light source carriage 902. In some other examples, the pivotal mounting 908 permits another other movement relative to the light detection arrangement 40. In some other examples, additionally or alternatively, the light detection arrangement 40 is configured to move relative to the light source 20. That is, the light detection arrangement 40 is optionally configured to tilt, pivot or rotate with respect to the light source 20.

In some other examples, the light source carriage 902 and the transverse rail assembly 900 are not used. Alternatively, the base 200 and the light source 20 are both mounted to the same rigid object e.g., a workbench.

In some examples, the light source 20 comprises a plurality of individual light sources 20 arranged parallel to the fourth axis 180 orthogonal to the transmission axis 30. In some examples, additionally or alternatively the light source 20 comprises a plurality of individual light sources 20 arranged parallel to the fifth axis 190 orthogonal to the transmission axis 30. This means that a plurality of light beams can be transmitted along the transmission axis 30. The plurality of light sources 20 can be identical or different. This can mean that the light source 20 comprises a greater power or e.g., can emit a plurality of different frequency light sources 20 or multiple polarizations at the same time or multiplexed in time.

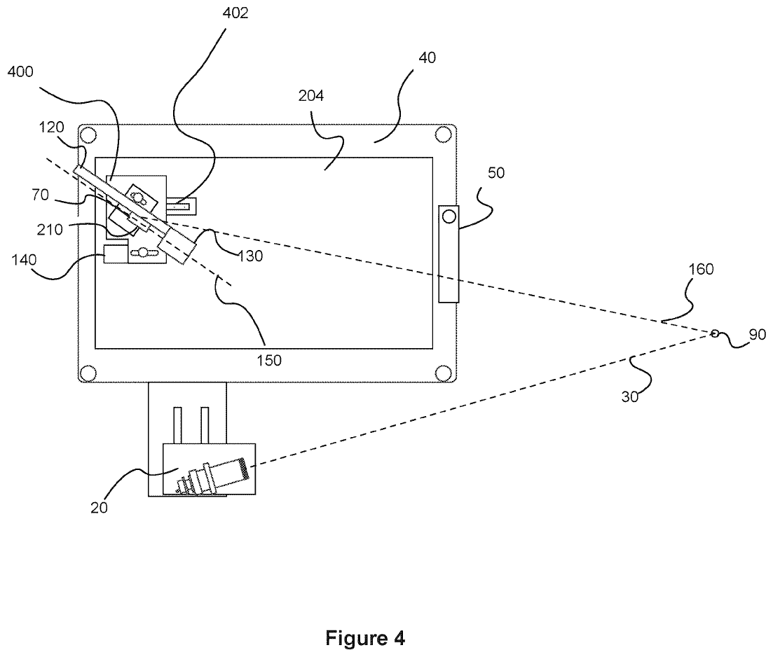

The light detection arrangement 40 will now be discussed in further detail with respect to FIGS. 3 and 4. FIG. 3 shows a schematic representation of the light detection arrangement 40. FIG. 4 shows a plan view of the light detection arrangement 40. The light detection arrangement 40 comprises a sensor assembly 120. The sensor assembly 120 is mounted to the base 200 via the component mounting plate 204 at a second end 208 of the base 200.

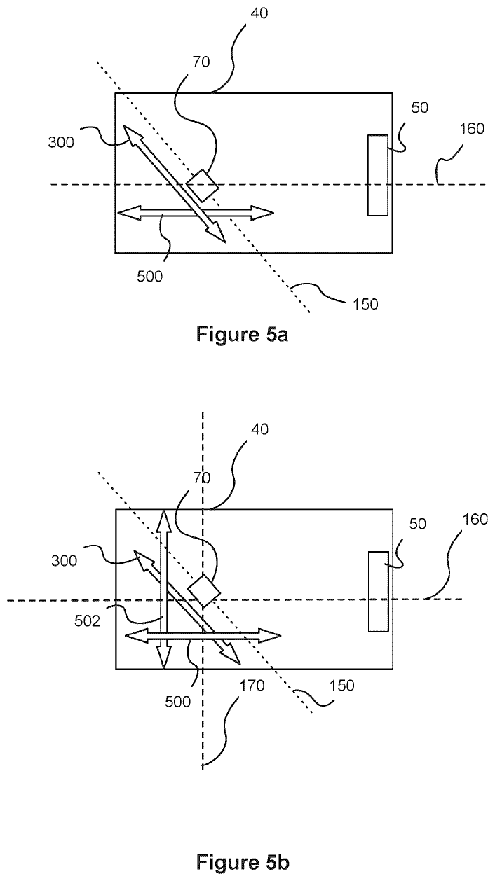

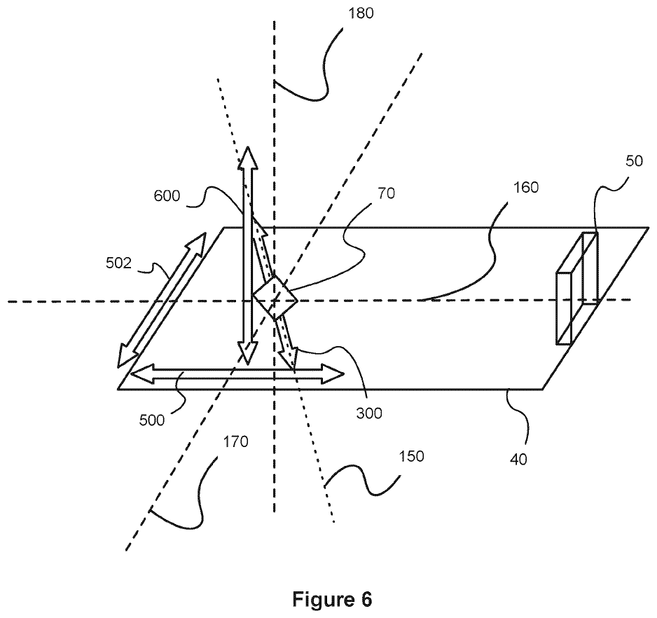

The sensor assembly 120 is mounted to the component mounting plate 204 via a sensor carriage 400. The sensor carriage 400 can be fixed with respect to the base 200. In some other examples as described below in reference to FIGS. 5a, 5b and 6, the sensor carriage 400 can be moveable with respect to the base 200.

Turning back to FIG. 4, the sensor assembly 120 comprises a sensor plate 212 (best shown in FIG. 7a) aligned in or parallel with the first axis 150. The plane of the sensor plate 212 is aligned with the first axis 150.

The light sensor 70 comprises a light sensor housing 210 which is slidably mounted along on the sensor plate 212 of the sensor assembly 120 via at least one sensor rail 700 e.g. a pair of sensor rails 700 (best shown in FIGS. 7a, 7b). The sensor plate 212 comprises a sensor window 702. The sensor window 702 is elongated and extends in a direction parallel with the first axis 150. The sensor window is also configured to intersect with the second axis 160. In this way, the returned scattered light will be received through the sensor window 702. The light sensor 70 faces the sensor window 702 towards the lens arrangement 50. The sensor window 702 as shown in FIG. 4 is separate from the light sensor 70. In some other examples, the sensor window 702 is integrated into the light sensor housing 210. In some examples, the sensor window 702 can be a circular hole rather than the elongate slot as shown in FIGS. 7a, 7b. In this case, the sensor window 702 is located in an additional plate (not shown) which is mounted to the light sensor housing 210. In this case, the sensor window 702 is fixed with respect to the light sensor 70 and both the sensor window 702 and the light sensor 70 move together along the first axis 150. In some examples, the sensor window 702 optionally comprises an adjustable slit component (not shown). The adjustable slit component is configured to change the position, size and/or orientation of an aperture between the light sensor 70 and the lens arrangement 50. This means that the adjustable slit component can select the amount of scattered light received by the light sensor 70. In some examples, the adjustable slit component is integrated into the sensor window 702. Alternatively, the adjustable slit component is a separate element mounted in front of the light sensor 70 or the sensor window 702.

The adjustable slit component comprises an adjustable aperture configured to allow adjustments of the slit width. In some examples, the slit width extends in a direction parallel with the first axis 150, and the height extends along a direction parallel with the fourth axis 180. Accordingly, the adjustable slit components can selectively adjust the width of the slit in the direction of the first axis 150. This means that adjustable slit component can be adjusted to allow the most relevant light signal to the light sensor 70. The adjustable slit component comprises a slit actuator (not shown) configured to adjust the size of the slit. The slit actuator can be operatively coupled to any suitable mechanism for adjusting the slit width. E.g. two moveable plates, an iris etc.

The light sensor 70 is mounted on the pair of sensor rails 700 such that the light sensor 70 is aligned with the sensor window 702. The light sensor 70 is configured to be aligned with the sensor window 702 when the light sensor 70 is moved with respect to the sensor plate 212. The pair of sensor rails 700 ensure that the light sensor 70 moves parallel to the first axis 150.

As mentioned above, the light sensor 70 is moveable via an actuator assembly 110. In a first example, the light sensor 70 is moveable along the first axis 150 with respect to the base 200. The movement of the light sensor 70 is schematically represented in FIG. 3 with arrow 300. The actuator assembly 110 comprises a first actuator 130 operatively connected to the light sensor housing 210 such that the light sensor 70 moves with respect to the sensor plate 212 when the first actuator 130 is actuated.

In some examples, the light sensor 70 and the light sensor housing 210 is mounted on a rod 704 having a helical thread mounted in a threaded bore 706 of the sensor plate 212. As the rod 704 rotates, the helical thread of the rod 704 moves in or out of the threaded bore 706 of the sensor plate 212. This causes the light sensor 70 to move along the first axis 150 either towards the first intersection 61 or away from the first intersection 61. This advantageously maintains the Scheimpflug condition 61. In some examples, the first actuator 130 is an electric motor (not shown) comprises a drive gear mounted to the drive shaft of the electric motor which is operatively coupled to the helical thread. Accordingly, rotation of the drive shaft of the electric motor causes the rod 704 to move and the position of the light sensor 70 is adjusted.

In some other examples, the first actuator 130 can be other types of actuators. For example, the first actuator 130 can be any other suitable linear actuator such as a pneumatic linear actuator, a hydraulic linear actuator, a linear servo, stepper motor, piezo motor or any other suitable linear actuating mechanism.

The arrangement as shown in FIGS. 3 and 4 represent the device 100 wherein the light sensor 70 is moveable along a single degree of freedom e.g., along the first axis 150. In some other examples, the light sensor 70 is moveable in a plurality of different directions in addition to being moveable along the first axis 150. FIGS. 5a and 5b show schematic arrangement of the device 100 wherein the light sensor 70 is moveable in more than one direction with respect to the light detection arrangement 40.

FIG. 5a shows the light sensor 70 being moveable along the first axis 150 and moveable along the second axis 160 or parallel to the second axis 160. The second axis 160 is the optical axis of the lens arrangement 50. The movement of the light sensor 70 and arrangement for providing the movement of the light sensor 70 along the first axis 150 is the same as described in reference to the previous Figures.

Movement of the light sensor 70 along the second axis 160 is provided by moving the sensor carriage 400 in a direction parallel to the second axis 160. The sensor carriage 400 comprises a projecting foot (not shown) which is slidable in a carriage slot 402. The carriage slot 402 restricts the relative movement of the projecting foot from the sensor carriage 400 in a direction along or parallel with the second axis 160 as shown by the arrow 500. The sensor carriage 400 is operatively coupled to a second actuator 140. The second actuator 140 is configured to move the sensor carriage 400 towards the first end 206 of the base 200 or towards the second end 208 of the base 200. The second actuator 140 in some examples is another linear actuator. In some examples, the second actuator 140 is identical to the first actuator 130. In some other examples, the second actuator 140 can be any other suitable linear actuator such as a pneumatic linear actuator, a hydraulic linear actuator, a linear servo, stepper motor, piezo motor or any other suitable linear actuating mechanism.

Since the light sensor 70 is configured to move along the first axis 150 and the second axis 160, the light sensor 70 can be moved in directions both parallel and perpendicular to the second axis 160. This means that the light sensor 70 can be moved to cover most of the light detection arrangement 40.

FIG. 5b shows an arrangement which is identical to the arrangement shown in FIG. 5a except that the light sensor 70 is configured to move along another direction. In this example, the actuator assembly 110 comprises a third actuator (not shown). The third actuator is configured to move the light sensor 70 along a third axis 170 or in a direction parallel to the third axis 170 as shown by the arrow 502. The third axis 170 is an axis which is perpendicular to the second axis 160 e.g., the optical axis of the lens arrangement 50. In some examples, a secondary sensor carriage (not shown) is mounted on the sensor carriage 400. The secondary sensor carriage is mounted to the sensor carriage 400 via a plurality of rails extending in a direction parallel with the third axis 170. The secondary sensor carriage is slidable with respect to the sensor carriage 400 along a direction parallel with the third axis 170. The third actuator is identical to the second actuator 140 and mounted on the sensor carriage 400.

FIG. 6 shows an arrangement which is identical to the arrangement shown in FIG. 5b except that the light sensor 70 is configured to move along another direction. In this example, the actuator assembly 110 comprises a fourth actuator (not shown). The fourth actuator is configured to move the light sensor 70 along a fourth axis 180 or in a direction parallel to the fourth axis 180 as shown by the arrow 600. The fourth axis 180 is an axis which is perpendicular to the second axis 160 e.g., the optical axis of the lens arrangement 50 and the third axis 170. In other words, moving the light sensor 70 in the fourth axis 180 adjusts the height of the light sensor 70 above the base 200. In some examples, the sensor carriage 400. The sensor plate 212 may be mounted to the sensor carriage 400 via a scissor linkage, worm gear motor, stepper motor, piezo motor and actuated by the fourth actuator. All of these types of actuators e.g. scissor linkage, worm gear motor, stepper motor, piezo motor can be locked into position, actively or passively. The sensor plate 212 is moveable with respect to the sensor carriage 400 along a direction parallel with the fourth axis 180 as the scissor linkage extends or retracts. The fourth actuator is identical to the second actuator 140 and mounted on the sensor carriage 400. Alternatively, any other suitable mechanism can be used to adjust the height of the light sensor 70 above the base 200.

In some examples the light sensor 70 comprises at least one of a single pixel, a quadrant of pixels, an array of pixels, a pixel matrix, a position sensitive device (PSD) pixel. The light sensor 70 may have at least one column of pixels aligned parallel to the first axis 150. The light sensor 70 comprising at least one row of pixels aligned parallel to the fourth axis 180.

In some examples, the light sensor 70 comprises at least one of a photodiode, avalanche photodiode, or photo multiplying tubes (pmt). The light sensor (70) may comprise at least one of a trans-impendence amplifier, free silicon amplifier, and dynode amplifier.

In some examples, the light sensor 70 is configured to detect a signal generated by at least one of wavelength modulation spectroscopy, direct absorption spectroscopy, and/or frequency modulation spectroscopy.

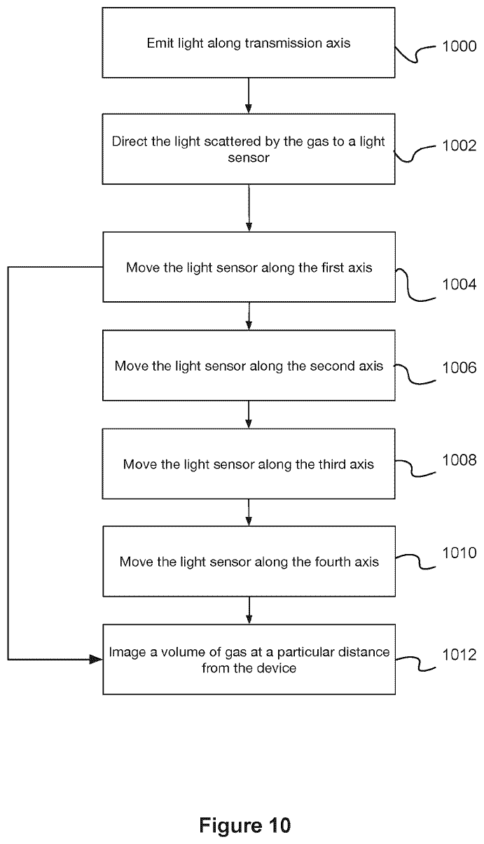

Operation of the device 100 will now be discussed in reference to FIG. 10. FIG. 10 shows a flow diagram of the method of operation of the device 100.

In an embodiment, data processing device 10 is configured to operate the device 100. The data processing device 10 sends a control signal to emit light along the transmission axis 30 as shown in step 1000.

The data processing device 10 is configured to position the light sensor 70 and/or the sensor window 702 and/or the sensor windows 702 (e.g. as shown in FIGS. 7a, 7b) to a defined position along the planes formed by the sensor axes as shown in steps 1004, 1006, 1008, 1010 and described in more detail below. The device 100 directs the light scattered by the gas to the light sensor 70 as shown in step 1002. In some examples, the light sensor 70 comprises a single moveable pixel. In this case, moving the sensor window 702 and/or the slit together with the light sensor 70 allows for better signal detection. Optionally, the slit width and the sensor positions are adjusted to optimize for the target volume in space, including range, individually.

The data processing device 10 is configured to image a volume of gas at a particular distance in step 1012. In step 1012, the data processing device 10 is optionally configured to process sensor signal 75 to determine signal S when the light source 20 is activated and determine background signal B when the light source 20 is not activated.

In step 1012, the data processing device 10 is also optionally configured to normalise signal S using background signal B. In one embodiment, background signal B is subtracted from signal S.

In step 1012, the data processing device 10 is also optionally configured to apply appropriate threshold and corrections for non-constant range dependency. The result of the normalising step is the intensity-as-a-function-of-sensor-position signal. This must be converted to intensity-as-a-function-of-range signal. Consequently, this step comprises the transformation of the raw intensity-as-a-function-of-sensor-position signal to an intensity-as-a-function-of-range signal.

In step 1012, the data processing device 10 is also optionally configured to process the intensity-as-a-function-of-range signal to determine the presence of gas absorption imprints by retrieving the baseline model available from either: the multimode setup looking at the light which is on resonance and off resonance with a gas of interest or through scanning a single-mode laser. The data processing device 10 can also optionally detect and analyse particles 90 as well.

In step 1012, the data processing device 10 is also optionally configured to correlate the results of the process the intensity-as-a-function-of-range signal step with previously determined results for noise reduction and/or to provide temporal information with respect to the results.

The data processing device 10 is then configured to repeat one or more of the steps as necessary.

As mentioned above, the data processing device 10 is configured to perform process steps for setting up the device 100 in order to move the light sensor 70 to the correct position in steps 1004, 1006, 1008 and 1010 which will be described in more detail.

The data processing device 10 is configured to send a control signal to the first actuator 130 to move the light sensor 70 along the first axis 150 as shown in step 1004 in FIG. 10. The data processing device 10 in some examples receives a sensor signal 75 from the light sensor 70 as the first actuator 130 moves the light sensor 70 along the first axis 150. The data processing device 10 sends a control signal to the first actuator 130 when the data processing device 10 determines that the light sensor 70 is in the correct position along the first axis 150. Movement along the first axis 150 corresponds to range of the detected light scattered back from the particle 90 towards a light detection arrangement 40. In some examples, the data processing device 10 sends the control signal to the first actuator 130 so that the light sensor 70 moves along the first axis 150. In other examples, the data processing device 10 moves the first actuator 130 throughout the entire range of movement of the first axis 150. The data processing device 10 then determines the intensity of the signal of detected light scattered back from the particle 90 as a function of range. In this way, the data processing device 10 is configured to determine the range of the particle 90 from the light detection arrangement 40.

Once the data processing device 10 has moved the light sensor 70 to the required position along the first axis 150, the data processing device 10 is then configured to image a volume of gas at a particular distance from the device 100 as shown in step 1012. If the data processing device 10 is configured to only move the light sensor 70 in the first axis 150, then the data processing device 10 goes from step 1004 to step 1012. This is shown by the arrow connecting steps 1004 and 1012.

Additionally, or alternatively, the data processing device 10 is configured to move the light sensor 70 in the second axis 160, the third axis 170 and/or the fourth axis 180. In this case the data processing device 10 proceeds from step 1004 to step 1006, step 1008 and step 1010. Furthermore, the data processing device 10 can skip to step 1012 from any of the steps 1006, 1008, 1010 without implementing any of the other steps 1006, 1008, 1010.

In some alternative examples, data processing device 10 is configured to continuously scan the light sensor 70 to get all ranges sequentially by moving the light sensor 70 along the first axis 150. In some examples, data processing device 10 is configured to adjust the slit width of the adjustable slit component to increase the spatial resolution at the interrogated volume at a particular distance.

In some examples, in steps 1002, 1004, 1006, 1008 or 1010, the data processing device 10 optionally sends a control signal to the slit actuator to fully open slit. This results in high signals but bad spatial resolution but the data processing device 10 can perform a coarse scan along the first axis 150. The data processing device 10 then determines that signal received at a narrow range is of interest. In this way the data processing device 10 is configured to “lock onto” a specific range of interest. The data processing device 10 may use one or more parameters of the received light signal to select a specific range of interest e.g. intensity, distance of the particle 90 etc. The data processing device 10 then sends a control signal to the slit actuator to narrow down the slit width. By narrowing the slit width, the data processing device 10 is configured to improve the range resolution whilst at the same time as increasing data collection time.

Optionally the data processing device 10 is then configured to send a control signal to the second actuator 140 to move the light sensor 70 along the second axis 160. Optionally the data processing device 10 is then configured to send a control signal to the third actuator to move the light sensor 70 along the second axis 160. Optionally the data processing device 10 is then configured to send a control signal to the fourth actuator to move the light sensor 70 along the fourth axis 180. The data processing device 10 is configured to control the movement of the second actuator 140, the third actuator and the fourth actuator in the same way as described for the first actuator 130.

The disclosure has mainly been described above with reference to a few examples. However, as is readily appreciated by a person skilled in the art, other examples than the ones disclosed above are equally possible within the scope and spirit of the disclosure, which is defined and limited only by the appended patent claims.

In another example, two or more examples are combined. Features of one example can be combined with features of other examples.

Examples of the present disclosure have been discussed with particular reference to the examples illustrated. However, it will be appreciated that variations and modifications may be made to the examples described within the scope of the disclosure.

1. A device for detecting a property of a gas comprising:
a light source configured to emit a light along at least a transmission axis,
a light detection arrangement comprising:
a light sensor configured to output a sensor signal,
a lens arrangement having a lens plane, and being configured to direct the light from the light source and scattered by the gas to the light sensor,
an actuator assembly configured to move the light sensor in a direction parallel to at least a first axis,
wherein the first axis, the transmission axis, and the lens plane intersect such that a Scheimpflug condition is achieved.

2. The device of claim 1, wherein the actuator assembly is further configured to move the light sensor in a direction parallel to a second axis, corresponding to an optical axis of lens arrangement.

3. The device according to claim 2, wherein the actuator assembly is further configured to move the light sensor in a direction parallel to a third axis orthogonal to the second axis and in a plane defined by the first axis and second axis.

4. The device according to claim 1, wherein the actuator assembly is configured to move the light sensor in a direction parallel to a fourth axis along a normal of the plane defined by the first axis and second axis.

5. The device of claim 1, wherein the light sensor is mounted on a sensor assembly.

6. The device of claim 5, wherein the light sensor is moveable with respect to the sensor assembly and the actuator assembly comprises a first actuator configured to move the light sensor.

7. The device of claim 5, wherein the sensor assembly is moveable with respect to a housing of the device and the actuator assembly comprises a second actuator configured to move the sensor assembly.

8. The device of claim 7, wherein the sensor assembly is moveable with respect to a housing of the device along a rail.

9. The device of claim 1, wherein the light sensor comprising at least one of a single pixel, a quadrant of pixels, an array of pixels, a pixel matrix, a position sensitive device (PSD) pixel.

10. The device of claim 1, wherein the light sensor comprising at least one column of pixels aligned parallel to the first axis.

11. The device according to claim 4, wherein the light sensor comprising at least one row of pixels aligned parallel to the fourth axis.

12. The device of claim 1, wherein the light sensor comprises at least one of a photo diode, an avalanche photodiode, a photo multiplying tube (pmt), and a cmos sensor.

13. The device of claim 1, wherein the light sensor comprises at least one of a trans-impendence amplifier, free silicon amplifier, current amplifier, and dynode amplifier.

14. The device of claim 1, wherein the light sensor being configured to detect a signal generated by at least one of wavelength modulation spectroscopy, direct absorption spectroscopy, and/or frequency modulation spectroscopy.

15. The device of claim 1, wherein light source is a tuneable diode laser.

16. The device of claim 15, wherein the light source is controlled in a TDLAS fashion.

17. The device of claim 15, wherein the light source is controlled in a DIAL fashion.

18. The device according to claim 4, wherein light source comprises an array of individual light sources arranged parallel to a fifth axis orthogonal to transmission axis or parallel to the fourth axis.

19. The device of claim 1, wherein the light sensor comprises a single sensor pixel.

20. The device of claim 1, wherein the device comprises a sensor window positioned between the light sensor, and the lens arrangement.

21. The device according to claim 20 wherein the sensor window comprises a slit having adjustable width in a direction along the first axis.

22. A method for detecting a property of a gas comprising:
emitting a light along at least a transmission axis,
directing the light scattered by the gas to a light sensor using a lens arrangement having a lens plane,
imaging a volume of gas at a particular distance from a device by moving the light sensor to a corresponding position along a first axis,
wherein the first axis, the transmission axis, and the lens plane) intersect such that a Scheimpflug condition is achieved.
